(19) United States
(12) Patent Application Publication
(10) Pub. No.: US 2014/0330219 A1
(43) Pub. Date: Nov. 6, 2014

(54) RELEASE DEVICE FOR RELEASING A MEDICAL IMPLANT FROM AN INSERTION DEVICE, AND ALSO INSERTION DEVICE COMPRISING A RELEASE DEVICE

(71) Applicant: BIOTRONIK AG, Buelach (CH)

(72) Inventor: Bodo Quint, Oberglatt (CH)

(73) Assignee: BIOTRONIK AG, Buelach (CH)

(21) Appl. No.: 14/261,223

(22) Filed: Apr. 24, 2014

Related U.S. Application Data
(60) Provisional application No. 61/819,674, filed on May 6, 2013.

(51) Int. Cl. A61F 2/95; A61F 2/24

(52) U.S. Cl. CPC A61F 2/95; A61F 2/2427; A61F 2002/9517
USPC 604/264

(57) ABSTRACT

A release device (100, 100a) for releasing a medical implant (105) from an insertion device (110), with which the implant (105) can be released by a relative movement between a first and a second insertion element (92, 94), the release device having a body (10, 10a) with a proximal end (12)and a distal end (14), wherein a hand grip unit (16) with a first grip segment (18) fixed relative to the body (10, 10a) and at least one movably arranged second grip segment (20, 20a) is provided between the proximal and the distal end (12, 14), wherein the movably arranged second grip segment (20, 20a) is rotatable in the to circumferential direction (22) in order to effect an intended relative movement between the first and the second insertion element (92, 94) of the insertion device (110).

### CROSS REFERENCE TO RELATED APPLICATIONS

This application claims benefit of priority to U.S. patent application Ser. No. 61/819,674 filed May 6, 2013; the content of which is incorporated by reference in its entirety.

### TECHNICAL FIELD

The invention relates to a release device for releasing a medical implant from a catheter and to a catheter comprising a release device for releasing a medical implant for implantation in an animal and/or human body according to the preambles of the independent patent claims.

### BACKGROUND

In the field of medicine, implants are often used that are introduced into an animal and/or human body either permanently or at least for a relatively long period of time in order to carry out replacement functions. For example, these implants could include heart pacemakers, brain pacemakers for Parkinson's patients, cardiac implants, such as cardiac valves or what are known as septum-closure devices, cochlear implants, retinal implants, dental implants, implants for joint replacement, vessel prostheses, for example insertable into the pulmonary vein, occluders, for example for the appendix, or stents.

Implants are connected to catheters before insertion into the body and have to be able to be placed precisely at the site for use and released in a defined manner. To this end, it is known for example to release the implant by a sliding motion.

A particular challenge is posed by large implants, which generally also require a more rigid delivery system in order to be released in an anatomy that changes substantially over time.

Here, a trauma may be produced by vessel contact and excessively slow release of the implant, or the intended placement of the implant may be shifted by the changing anatomy. This is true in particular for minimally invasively implantable cardiac valves, that is to say what are known as “appendix closure devices”, and for the placement of nitinol stents in the pulmonary veins.

### SUMMARY

The object of the invention is to disclose a release device with which the intended release of an implant is improved.

A further object can be considered that of providing a corresponding insertion device.

The object is achieved in accordance with the invention by the features in the independent claims. Favorable embodiments and advantages of the invention will emerge from the other claims and from the description.

A release device for releasing a medical implant from an insertion device is proposed, with which the implant can be released by a relative movement between a first and a second insertion element. The release device includes a body with a proximal end, which is remote from a distal end of the insertion device during use, and with a distal end, which faces the distal end of the insertion device during use, wherein a hand grip unit with a first grip segment fixed relative to the body and at least one movably arranged second grip segment is provided between the proximal and the distal end, wherein the movably arranged second grip segment is rotatable in the circumferential direction in order to effect an intended relative movement between the first and the second insertion element of the insertion device.

As a result of the embodiment according to the invention, a release device can be provided that can be operated intuitively. Furthermore, the rotatable second grip segment or the rotary mechanism can be grasped by an entire hand. In addition, it contains at least two functions. By rotating the second grip segment, a slow, metered release of the implant is controlled, and by pulling on the second grip segment the implant is released directly and quickly. This is particularly user-friendly, since it can be implemented for the operator, in particular by grasping with both hands. Furthermore, the fixing hand, in particular engaging the first grip segment, can also always retain its function of positioning of a delivery system, such as a catheter, relative to the treated patient. Since the hands as a whole can be used and functions are not limited to individual fingers in terms of operation, large forces can be introduced manually. Furthermore, by means of the rotary movement, geared-down powerful movements can be transferred, as a result of which the implant can be reliably and homogeneously released and also positioned precisely and carefully. The implant can thus be released powerfully and/or gradually and also carefully. In addition, such a simple concept that can also be realized quickly in practice for releasing the implant is implemented. The partly released implant can particularly advantageously be repositioned or withdrawn by means of the embodiments according to the invention, for example if positioned incorrectly. This is particularly the case since the rotary movement can produce a large force for again compressing the partly expanded implant. Furthermore, the release device can be easily handled and allows the implant to be easily assembled on the insertion device, for example a catheter, in the preparation laboratory. The concept according to the invention is particularly well suited and can be used advantageously for a moving anatomy, such as the heart.

In this context, a “grip unit” is to be understood to mean a unit that is handled, grasped and/or operated with at least one hand by an operator, such as a technician, a doctor or a carer, in particular in the event of assembly of the implant in the laboratory or a release of the implant during implantation in the body. A “grip segment” is to be understood to mean a portion or region of the grip unit that differs with regard to at least one parameter, such as a placement, a function, an operating mechanism and/or another parameter considered appropriate by a person skilled in the art, from at least one other portion or region of the hand grip unit. The second grip segment preferably extends in the circumferential direction at least 50%, advantageously at least 75%, and particularly preferably at least 100% over a circumference of the body. The first grip segment is advantageously arranged fixedly relative to the body or the component parts inside the body and/or is formed in one piece with the body or the housing thereof, wherein the term “in one piece” is to be understood to mean that the first grip element and the body are formed by the same component part and/or can be separated from one another merely with a loss of function of at least one of the component parts. Furthermore, it is arranged distally from the second grip segment. Here and in the following text, the term “effect” is to be understood to mean “produce, prompt and/or achieve”.

The second grip segment advantageously conveys the relative movement between the first and the second insertion element via at least one effective connection to a thread. The ergonomically high forces that can be easily produced by the rotary movement of the second grip segment can be transferred particularly uniformly by the use of the thread. In addition, a precise and delicate manipulation is possible, even in spite of a possible use of a high thread pitch of the “thread transfer” for the gearing down. In this context, an “effective connection” is to be understood in particular to mean an interlocking connection and/or a force-locked connection. In this context, a “thread” is to be understood to mean an effective element that conveys and/or transfers the effect of the second grip segment. In particular, it is also to be understood to mean a spindle or a screw. The thread is preferably fixed relative to the first grip segment or is arranged or formed in/on a component part, such as a main body, which is arranged fixedly relative to the first grip segment. In addition, the thread is fixed relative to an insertion element and in particular to an inner insertion element, and for example is fixed relative to the inner shaft in the case of a catheter.

Furthermore, it is proposed for the effective connection between the second grip segment and the thread to be produced via at least one coupling element, whereby the transfer can be variable. The coupling element may be formed by any element considered usable by a person skilled in the art, such as a pin, a roller, or preferably a ball. In addition, the coupling element is designed in an advantageous embodiment as a following, in particular axially following, coupling element. The coupling element is advantageously held in a cage, in particular an axially movable cage. If, for example, the coupling element is formed by a ball, the cage is formed as a ball cage. Furthermore, it is advantageous if the effective connection between the second grip segment and the thread constitutes at least one force-locked connection, whereby forces can be transferred in a constructionally simple manner. Alternatively and/or in addition, it would also be conceivable for the effective connection to constitute an interlocking connection, whereby a stable effective connection can be formed.

In an alternative embodiment of the invention, it is proposed for the effective connection between the second grip segment and the thread to be provided via at least one toothing element. A particularly reliable and insusceptible connection can thus be ensured. The toothing element may be formed by any element considered usable by a person skilled in the art, such as a gearwheel, a spindle, a screw, or preferably a toothed rack.

It is also proposed for the relative movement between the first and the second insertion element to be transferrable to one of the insertion elements by means of a connection element. A transfer can thus be designed in a particularly versatile manner. Furthermore, properties such as a material, a material hardness, an orientation and/or another property considered appropriate by a person skilled in the art of each component part can be matched to its individual function, without impairing a function of another component part. In this case, the insertion element is preferably an outer insertion element or, for example in the case of a catheter, an outer shaft. Furthermore, it may be advantageous if the connection element is effectively connected to the second grip segment, whereby the movement can be transferred reliably. In this context, the term “effectively” is to be understood to mean that the effect of the second grip segment can be conveyed or transferred via the connection between the connection element and the second grip segment.

In addition, a secure placement of component parts, in particular also relative to one another, can advantageously be achieved if the thread or the component part with the thread (the main body) is fixed by means of at least one fixing element. In this case, the fixing element may be formed by any component part considered usable by a person skilled in the art, such as a pin, a hook, a wall, or a plate. In addition, it is proposed for the fixing element to extend substantially radially, preferably radially, in relation to an inner axis of the insertion device. The fixing can thus be designed in a particularly space-saving manner. Here, “radially” is to be understood to mean perpendicularly. Furthermore, “substantially radially” is to be understood to mean a deviation of the direction of the fixing element relative to the direction of the inner axis of up to 30° from the radial or perpendicular arrangement. The fixing element is advantageously arranged on the first grip segment, whereby the stationary arrangement can be produced in a constructionally simple manner.

The fixing element alternatively and/or additionally advantageously extends substantially axially, preferably axially, in relation to the inner axis of the insertion device. The function of guiding an axially displaceable connection element can thus be assisted. Here, “axially” is to be understood to mean parallel to the catheter axis. Furthermore, “substantially axially” is to be understood to mean a deviation of the direction of the fixing element relative to the direction of the inner axis of up to 30° from the axial or parallel arrangement.

A preferred development lies in the fact that the connection element has at least one recess, in which the at least one fixing element engages. A position of the connection element can thus be reliably secured. The recess is preferably penetrated by the fixing element. The engagement of the fixing element in or through the recess is used in particular to fix the connection element in the peripheral direction. In particular, there is thus no transfer of the rotary movement of the second grip segment to the connection element and therefore to the insertion element. As a result, damaging stresses and twisting of the insertion element are avoided. In addition, the fixing element, in particular on account of its axial extension, can be used as a guide means, in particular an axial guide means, of the recess in the connection element in the event of an axial movement of the connection element during the relative movement between the first and the second insertion element of the insertion device, for example in the event of the release of the implant, whereby this relative movement can occur in a trouble-free manner.

In a preferred embodiment the connection element is coupled and/or connected to a transfer element for the effective connection to the second grip segment. The elements and the different functions thereof can thus also be decoupled. The transfer element comprises at least the cage for the coupling element. The transfer element is preferably connected to the connection element via an interlocking connection and/or it engages via an extension, which points radially inwardly and extends in the circumferential direction, in a groove in the connection element, which is open radially outwardly and likewise extends in the circumferential direction. A rotation of the two component parts relative to one another is thus possible, or the transfer element is arranged rotatably relative to the connection element. The transfer element can be rotated by the second grip segment, but a rotation of the connection element is prevented by the fixing element.

It is also proposed for the second grip segment to be biased in its axial rest state by at least one spring element, whereby the second grip segment can be held securely in position. In this case, an “axial rest position” is to be understood to mean a fixed position in the axial direction. Here, a “spring element” is to be understood to mean any resilient and/or elastic element, and in particular a spring, for example in the form of a compression spring. To implement this embodiment according to the invention, the second grip segment on its inner face has a cavity, in which a radially outwardly pointing extension of the transfer element engages. The spring element is fitted axially between the extension and a wall of the cavity in the second grip segment.

In accordance with an advantageous embodiment, the effective connection between the second grip segment and the thread can be released by a movement of at least one unlocking element. As a result, a further operating mode of the release device can be implemented in a constructionally simple manner. Here, an unlocking element may be formed by any component part considered practicable by a person skilled in the art, such as a rotary element, a tilting element and preferably a slide element and/or pull element. The unlocking element is preferably arranged directly on the second grip segment, whereby it is possible to switch easily and quickly between operating modes, for example using the same hand. Manual grasping can thus be omitted in a process-streamlining and time-saving manner. The effective connection is advantageously releasable against a spring tension of at least one spring element, whereby the activation force necessary to switch between various operating modes or between the rotary movement of the second grip segment and the push and/or pull movement of the unlocking element can be metered by technical means. For example, the spring element may be the spring element that biases the second grip element in its axial position or a further spring element, which may be arranged at any other point considered appropriate by a person skilled in the art, for example on the unlocking element itself

The release of the active connection advantageously effects a fast release of the implant, whereby the implant can be released reliably and quickly advantageously for a patient. This may be expedient and advantageous in particular with large implants and high release forces. By means of the rotary movements of the second grip segment, geared-down, powerful movements can be transferred, which are transferable directly in the event of the fast release by a push and/or pull movement. Such an embodiment according to the invention enables intuitive operation. Intensive training of the operator can thus be omitted in a time-saving manner. In addition, the operator can decide without delay between a sensitive release by means of rotation of the second grip segment and definitive release that is as spontaneous as possible by means of the unlocking element. The fast release can thus be initiated directly and unimpeded by additional operating measures. A sliding and smooth release of the implant is made possible by means of the push and/or pull movement of the unlocking element. In addition, two-handed operation or a changing of hands when switching to fast release is omitted as a result of the arrangement of the unlocking element on the second grip segment. A damaging change to the position of the implant can thus be prevented so as to improve the implantation process.

A key advantage however has been found to be the possibility of releasing the implant in a manner that is as spontaneous as possible in a situation in which time is critical. This decision and response may be important for example for the placement of a cardiac valve. The reason for this is the contact with a moving anatomy, which, from identification of the correct positioning of the implant to ultimate release, may lead to trauma, but also to displacement of the implant.

It is also proposed for the at least one coupling element to be moved radially away from an inner axis of the insertion device as a result of the movement of the at least one unlocking element and to thus be disengaged from the thread. As a result, the decoupling can be implemented in a constructionally simple and quick manner. With a construction with a coupling element and cage, the coupling element in this instance may preferably move away into a seat that is arranged and/or formed in the inner face of the second grip segment. With the construction with the toothing element, this may be removed from the interlocking connection to the thread, for example with pulling or pushing.

Alternatively and/or additionally, at least part of the unlocking mechanism or the entire unlocking mechanism and/or the unlocking element can also be blocked locally, independently of the release position, for example the function of the fast release.

More preferably, the at least one unlocking element is formed in one piece with the second grip segment, whereby space, assembly effort and costs can be saved. In this instance, the phrase “in one piece” is to be understood to mean that the unlocking element and the second grip segment are formed by the same component part and/or can be separated from one another only with a loss of function of at least one of the component parts.

In an alternative or additional embodiment, the unlocking element may be formed by a component part formed independently of the second grip segment. It preferably comprises a ring, which is arranged axially displaceably and in particular in the peripheral direction around the second grip segment. The unlocking element is advantageously arranged in an indentation in the second grip segment, wherein the unlocking element can be arranged in a space-saving manner. In addition, the second grip element and therefore the outer insertion element can thus be moved axially directly after unlocking using the same hand that operated the unlocking element.

In a further embodiment of the invention, it is proposed for the body, in particular the component part with a thread (the main body), to have an aperture for one of the insertion elements. This allows a compact arrangement, which stabilizes and protects the insertion element passed through. If the insertion device is a catheter, the insertion element in question may thus be an inner shaft of the catheter.

In accordance with an advantageous embodiment, the implant may be a self-expanding implant, whereby it can open automatically upon release. Due to the self-expanding implant, an additional expanding means can be omitted. Space and assembly effort for this can therefore be saved advantageously. The insertion device can thus also be formed in a less complex manner. In principle however, it would also be possible to use a balloon-expandable implant. To this end, the insertion device would have to be adapted accordingly however, which a person skilled in the art achieves independently on the basis of his common general knowledge in the art.

In accordance with a further aspect of the invention, an insertion device for inserting the medical implant, which can be released by a relative movement between the first and the second insertion element, is proposed and comprises the release device for releasing the medical implant, the release device including the body with the proximal end, which is remote from the distal end of the insertion device during use, and with the distal end, which faces the distal end of the insertion device during use, wherein a hand grip unit with a first grip segment fixed relative to the body and at least one movably arranged second grip segment is provided between the proximal and the distal end, wherein the movably arranged second grip segment is rotatable in the circumferential direction in order to effect an intended relative movement between the first and the second insertion element of the insertion device.

As a result of the embodiment according to the invention, an insertion device can be provided that can be operated intuitively. By means of the rotary movement, geared-down powerful movements can be transferred, as a result of which the implant can be reliably and homogeneously released and also positioned precisely and carefully. The partly released implant can particularly advantageously be repositioned or withdrawn by means of the embodiments according to the invention, for example if positioned incorrectly. This is particularly the case since the rotary movement can produce a large force for again compressing the partly expanded implant. Furthermore, the insertion device allows the implant to be easily handled and assembled in the preparation laboratory.

The invention will be explained in greater detail hereinafter by way of example with reference to exemplary embodiments illustrated in drawings, in which:

### DESCRIPTION OF THE DRAWINGS

FIG. 1 shows a schematic illustration of a section through a favorable exemplary embodiment of an insertion device and a release device with a partly released implant;

FIG. 2 shows a schematic illustration of a detail of a section through the release device in FIG. 1;

FIG. 3 shows a schematic illustration of a further detail of a section through the release device in FIG. 1;

FIG. 4 shows a schematic illustration of a section through the release device in FIG. 1 during an operating mode of fast release of the implant; and

FIG. 5 shows a schematic illustration of a section through an alternative release device.

### DETAILED DESCRIPTION

In the figures, functionally like or similarly acting elements are denoted in each case by like reference signs. The figures are schematic illustrations of the invention. They do not show specific parameters of the invention. The figures also merely reproduce typical embodiments of the invention and are not intended to limit the invention to the embodiments illustrated.

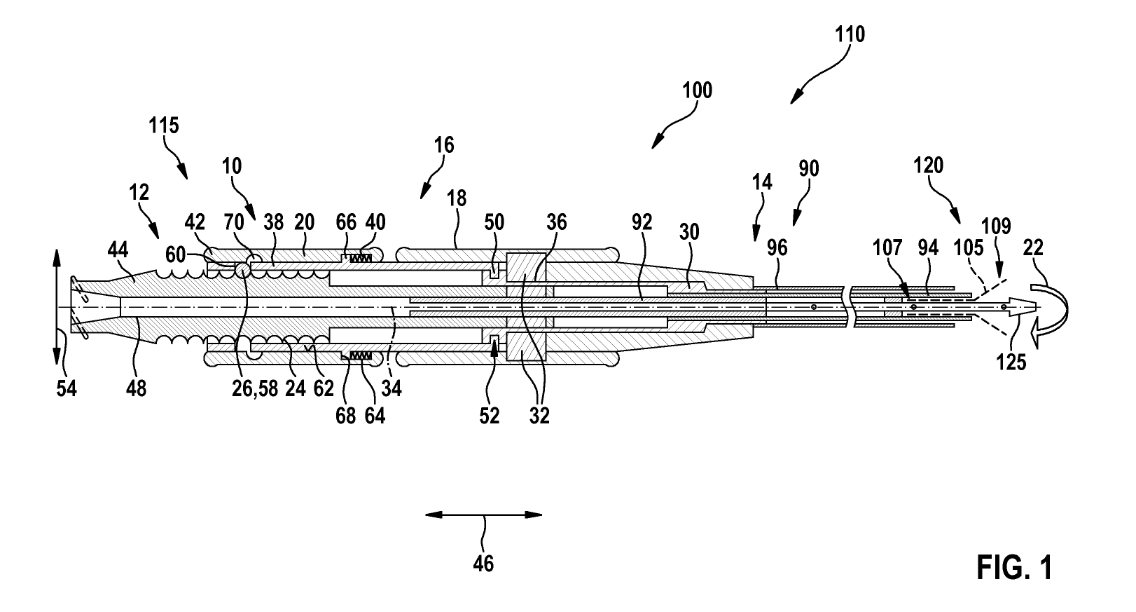

FIG. 1 shows a longitudinal section through a favorable exemplary embodiment of a release device 100 for a medical implant 105 of an insertion device 110 illustrated merely in part. For example, the insertion device 110 is a catheter having a shaft region 90 with two coaxially arranged insertion elements 92, 94, for example an inner shaft or what is also known as a “pusher tube” (insertion element 92) and an outer shaft or what is known as a “release tube” (insertion element 94), which surrounds the inner shaft and in turn can be surrounded by an outer sleeve 96 or what is known as a support tube. The outer sleeve 96 has the function of stabilizing the insertion elements 92, 94 during the insertion of the insertion device 110 into a patient and during a feed of the implant 105 to a site of implantation. In this case, the outer sleeve 96 helps the outer insertion element 94 to pass a haemostatic port or an introducer of an insertion system (not shown). On account of the outer sleeve 96, a release movement can be decoupled from the insertion system and conveyed neutrally through the insertion system, whereby a relative movement of the release tube is not transferred in the event of a release of the implant 105 to the port region and the haemostatic port. In addition, friction in the insertion system can thus be reduced. Due to the outer sleeve 96, a coupling to the patient may also be produced, which is used after positioning of the implant 105 as a fixing point for the position of the insertion device 110. This may also be assisted by a closure of the haemostatic port. The pusher tube (insertion element 92) is rigidly connected to the outer sleeve 96 and lengthens the shaft region 90 as far as a catheter tip 125. Furthermore, the inner insertion element 92 is connected to the catheter tip 125, but by contrast the outer insertion element 94 is not.

Since friction in the vessel is generally rather low, it is possible in principle to delimit the dimensions of the outer sleeve 96 merely to the port region or a region of tube-like insertion aids. In principle, in particular when not absolutely necessary for the placement of the implant, the release device may also be designed without an outer sleeve. For example, this is usually the case with release devices for implants manufactured on the basis of nitinol. In this case, the friction of the outer sleeve 96, which may lead to a displacement of the catheter, is accepted. However, it is possible to benefit from a reduced profile of the catheter obtained as a result. The insertion profile of the catheter is normally directly correlated by the user with the trauma for the patient associated with the minimally invasive intervention.

The proximal end 115 of the insertion device 110 faces a user during use, that is to say during a fastening of the implant 105 to the release device 100 or during implantation. The implant 105 is placed at the distal end 120 of the shaft region 90 between the inner shaft and outer shaft and is to be released at the site of implantation in the animal or human body.

The release device 100 is used to release the medical implant 105 from the insertion device 110. The implant 105 is arranged at the end 120 of the shaft region 90 remote from the user, for example in the vicinity of the catheter tip 125. The implant 105 is placed for example around the inner insertion element 92 and is released by a relative movement between the first and the second insertion element 92, 94. For release, the release tube (insertion element 94) is drawn “proximally” or in the direction of the proximal end 115 and, provided a distal end 109 of the implant 105 is arranged outside the outer sleeve 96, thus releases the implant 105.

The release device 100 comprises a body 10 with a proximal end 12, which is remote from the distal end 120 of the insertion device 110 during use, and with a distal end 14, which faces the distal end 120 of the insertion device 110 during use. A hand grip unit 16 is provided between the proximal and the distal end 12, 14 of the body 10. This hand grip unit 16 comprises a first grip segment 18 and a second grip segment 20. The first grip segment 18 is fixed relative to the body 10 or relative to component parts that are arranged inside the body 10, such as a main body 44, a fixing element 32 or a proximal region of the inner insertion element 92. For example, the first grip segment 18 may be formed in one piece or as a component part with the body 10 or a housing of the body 10. In this case, the release device 100 and the insertion device 110 are designed such that a position of the body 10 and of the first grip segment 18 relative to the implant 105 and the catheter tip 125 can be changed. The body 10 is connected via the first grip segment 18 to the outer sleeve 96. The second grip segment 20 extends in the circumferential direction 22 completely around a circumference of the body 10 and is arranged movably relative to the body 10 and the first grip segment 18. In addition, the first grip segment 18 is arranged distally with respect to the second grip segment 20 or in the direction of the distal end 14 of the body 10. The movably arranged second grip segment 20 is rotatable in the circumferential direction 22, whereby the intended relative movement between the first and the second insertion element 92, 94 of the insertion device 110 is effected.

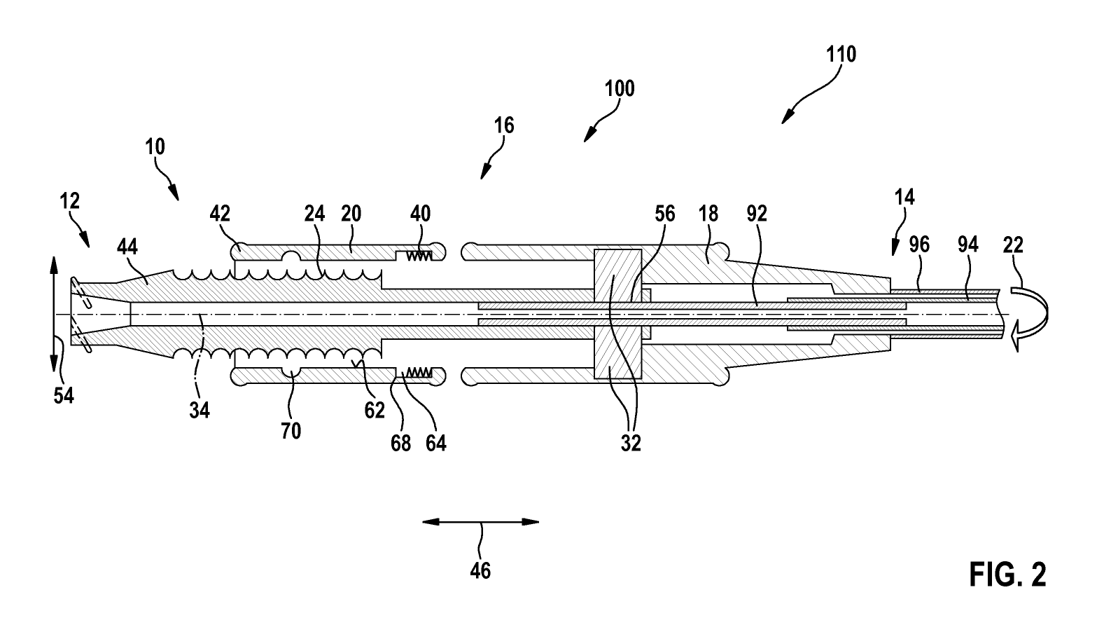

As can also be seen in detail in FIG. 2 (not all component parts are sketched in FIGS. 2 to 4 for improved clarity), the body 10 additionally comprises a main body 44 or catheter body, which extends in the axial direction 46 along an inner axis 34 of the insertion device 110 and entirely through each of the first and second grip segment 18, 20. The main body 44 is connected to the inner insertion element 92. The body 10 or the catheter body also has an aperture 48 for the inner insertion element 92. The inner insertion element 92 provides a lumen, for example for a guide wire (not shown). Furthermore, a Luer-Lock for possible flushing of the inner insertion element 92 is attached to the proximal end 12 of the body 10 or of the main body 44.

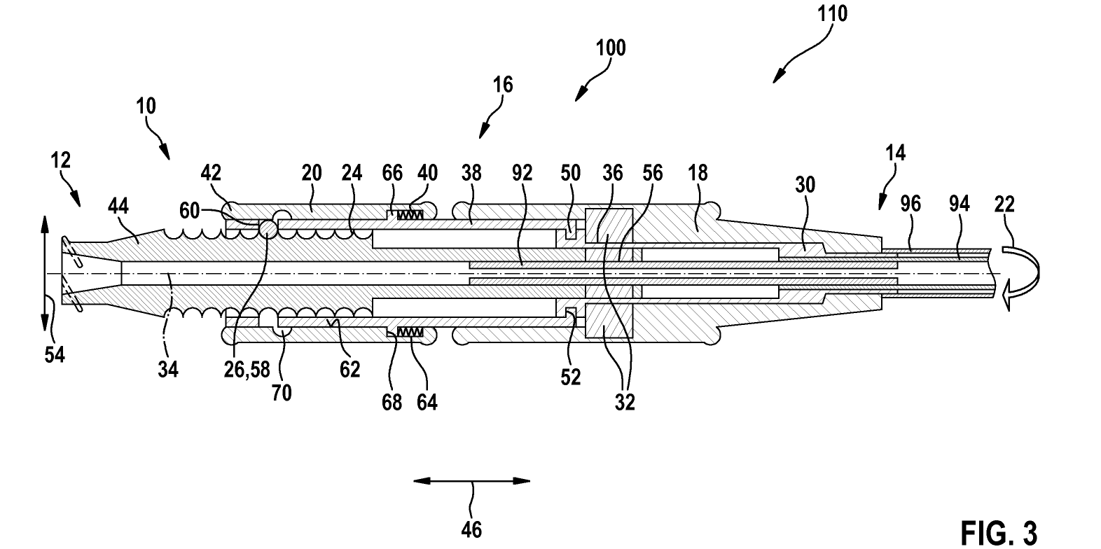

To transfer the relative movement between the inner and outer insertion element 92, 94, the body 10 additionally comprises a transfer unit with a transfer element 38 and a connection element 30. The transfer element 38 is connected via an interlocking connection to the connection element 30, wherein the transfer element 38 is arranged rotatably relative to the connection element 30 (see below). For this purpose, the transfer element 38 comprises an extension 50, which points radially inwardly toward the inner axis 34 and extends in the circumferential direction 22. This extension 50 engages in a groove 52 in the connection element 30, the groove being radially outwardly open for the engagement of the extension 50 and likewise extending in the circumferential direction 22. The transfer element 38 is arranged in the radial direction 54 radially between the second grip segment 20 and the main body 44, whereas the connection element 30 is arranged in the radial direction 54 radially between the first grip segment 18 and the main body 44. The connection element 30 is connected to the outer insertion element 94 at the distal end 14 of the body 10 (see FIG. 3).

On the first grip segment 18, two fixing elements 32, in the form of plates, are arranged fixedly relative to the first grip segment, wherein the two fixing elements 32 are arranged radially opposite one another and extend toward the inner axis 34 as far as the main body 44. For this purpose, the connection element 30 comprises recesses 36, which are arranged in the circumferential direction 22 at the same height as the fixing elements 32. The fixing elements 32 engage in the recesses 36 or the recesses are penetrated by the fixing elements 32. As a result, the connection element 30 is fixed in the circumferential direction 22. On the main body 44, the fixing elements 32 engage in depressions 56 and thus fix the main body 44 both axially and in the circumferential direction 22 relative to the first grip segment 18 (see FIG. 2). A rigid, fixed coupling between the first grip segment 18, with the fixing elements 32 and also the outer sleeve 96, and the main body 44, with the inner insertion element 92, is thus produced.

In order to convert the rotation of the second grip segment 20 into a relative movement between the outer and inner insertion element 92, 94 or an axial movement, the main body 44 includes, in the region of the second grip segment 20 or at its proximal end 12, a thread 24 extending in the circumferential direction 22. The main body 44 therefore functionally connects the thread 24 to the inner insertion element 92. The second grip segment 20 conveys the rotary movement via an effective connection with the thread 24, wherein the effective connection constitutes a force-locked connection, which is produced via a coupling element 26. The coupling element 26 is formed by a ball 58, which is matched in terms of its dimensions to the dimensions of the thread 24. For placement of the coupling element 26 radially between the second grip segment 20 and the main body 44 or the thread 24, the transfer element 38 comprises a cage or ball cage 60 in the form of an opening, in which the ball 58 is arranged.

The coupling element 26 or the ball 58 is held in the ball cage 60, since the second grip segment 20 is biased in its axial rest state by two spring elements 40 in the form of compression springs and thus radially covers the coupling element 26. For placement of the spring elements 40, the second grip segment 20 has one cavity 64 per spring element 40 on an inner face 62. In this case, the cavities 64 are arranged radially opposite one another. For axially fixed rest positioning on the transfer element 38 and for coupling, fixed against rotation, to the transfer element 38 and also for transfer of the rotary movement of the second grip segment 20 to the transfer element 38, the transfer element has two radially outwardly pointing extensions 66, each of which engages in a respective one of the cavities 64 in the second grip segment 20. The spring elements 40 are then each fitted axially between the extension 66 and a wall 68 of the cavity 64 in the second grip segment 20.

The mechanism of the release device 100 for the relative movement between the inner and outer insertion element 92, 94 by rotating the second grip segment 20 for a slow release of the implant 105 will be described hereinafter. This is the case for example when the operator expects the catheter to be correctly positioned and to “remain in place”. He thus tests the positioning of the implant 105 by means of a careful release. In this case, the second grip segment 20 is then rotated in the circumferential direction 22, and in so doing entrains the coupling element 26 or the ball 58 due to the effective or force-locked connection on the axially fixed thread 24. As a result, the coupling element 26 and the transfer element 38 are moved axially and, depending on the orientation of the thread and direction of rotation, in the direction of the proximal or distal end 12, 14. Due to the force-locked connection between the transfer element 38 and the connection element 30, the connection element is also moved axially together with the outer insertion element 94. In this case, the rotary movement is not transferred to the connection element 30, since this is fixed in the circumferential direction 22 by the fixing elements 32. The fixing elements 32 also enable an axial guidance of the movement in combination with the recesses 36. In addition, the fixing elements 32 also ensure a fixed arrangement of the thread 24 in the circumferential direction 22 and axial direction 46 via the engagement in the depressions 56 in the main body 44. Lastly, an axial movement, which moves the outer insertion element 94 relative to the inner insertion element 92 and the outer sleeve 96, is produced by rotating the second grip segment 20 over the spherical circumferential thread of the main body 44.

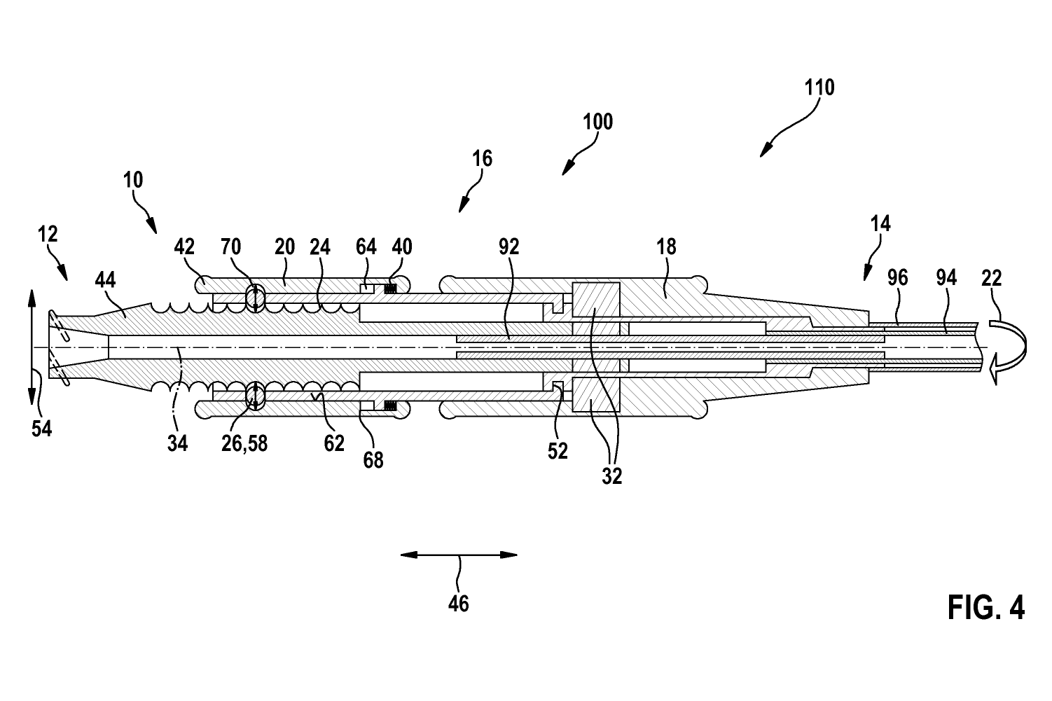

The release device 100 additionally comprises a mechanism for a fast release of the implant 105, as is illustrated in FIG. 4. In this case, the effective connection between the second grip segment 20 and the thread 24 can be released by a movement of an unlocking element 42 against the spring bias of the spring elements 40. The unlocking element 42 is formed in one piece with the second grip segment 20 or is formed thereby. For the fast release, the second grip segment 20 is then drawn for example in the direction of the proximal end 12 of the body 10, which appears intuitive for an operator, since in this case the outer insertion element 94 is “pulled off” from the implant 105. As a result of this movement of the unlocking element 42 or of the second grip segment 20, the coupling element 26 is moved radially away from the inner axis 34 of the insertion device 110 and therefore becomes disengaged from the thread 24. This occurs since the coupling element 26 can move away into a seat 70, which is formed on the inner face 62 of the second grip segment 20. An axial pull can thus be transferred at any time to the outer insertion element 94, without having to switch in a complex manner an operating state in advance or without an operator having to grasp the hand grip unit 16, since the hand responsible for the outer insertion element 94 already engages the second grip segment 20.

For implantation of the implant 105 in the body, the insertion device 110 or the insertion elements 92, 94 is/are flushed for the elimination of air therefrom in a first step. For this purpose, a syringe (not shown) is connected to the Luer-Lock of the main body 44 and a flushing medium, such as physiological saline solution, is introduced via the syringe into the insertion element 92 and via openings therein into the outer insertion element 94. The insertion device 110 provided with the implant 105 and thus prepared is then inserted into the body. After an initial placement of the distal end 120 of the insertion device 110 at the site of implantation, the implant 105 is released slowly by means of rotation of the second grip segment 20. As a result, the distal end 109 of the implant 105 is first released and expands automatically due to the design of the implant 105 as a self-expanding implant, such as a stent. If an exact positioning of the implant 105 is achieved, which can be monitored by a monitoring device, such as an X-ray device, the implant 105 as a whole or a proximal end 107 is released by means of the fast release and is completely opened due to its radial force. The release device 100 with the inner shaft is then withdrawn into the outer shaft and the insertion device 110 is removed from the body. The implant 105 remains fully positioned in the body (not shown).

In principle, both the mechanism of slow release and also of fast release can be used for assembly of the implant 105 on the insertion device 110. This is particularly possible since, due to the “gearing down” of the movement, high forces can be produced. In this case, the implant 105 is fastened on an exposed region of the inner insertion element 92 at the distal end 120 of the insertion device 110, for example by means of a crimping process. The implant 105 is then covered by the outer insertion element 94. For this purpose, the second grip segment 20 is either rotated in the opposite direction, as is the case in the event of the release, or is pushed in the direction of the distal end 14 of the body 10. Here, the process is started from a position of the second grip element 20, in which the grip element is in the proximally rear, fast release position. In order to prevent here a rash axial movement, conveyed by the spring element 40 compressed in this position, before the fastening of the implant 105 to the inner insertion element 92, a locking element (not shown in greater detail here) can be provided on the second grip segment 20. The pushing movement is assisted here by the compressed spring element 40. Once the implant 105 is covered by the outer insertion element 94, the assembly process is complete.

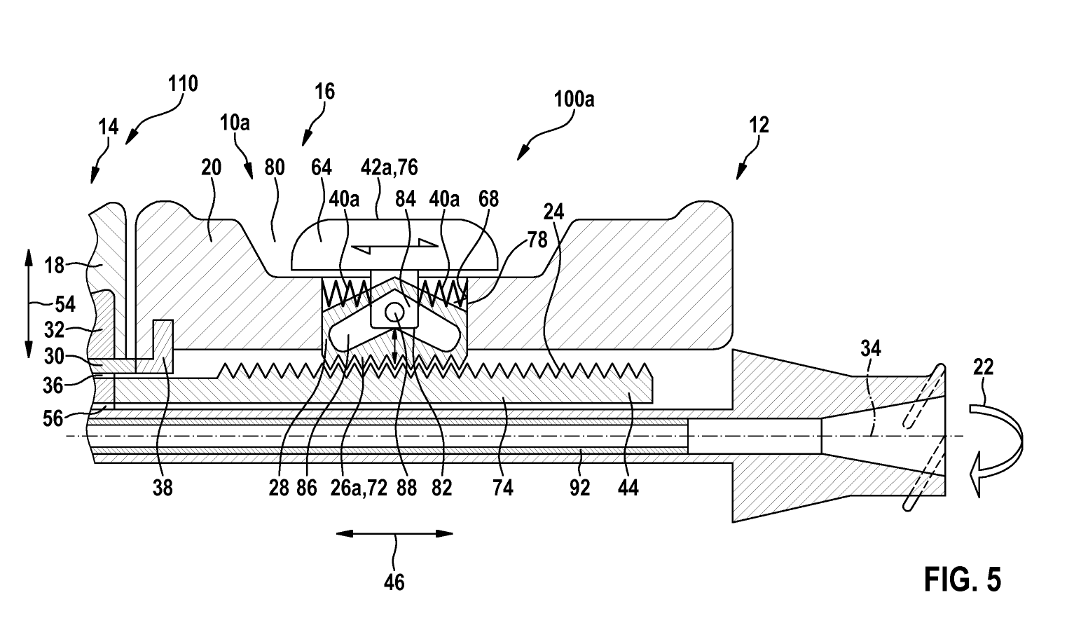

An alternative exemplary embodiment of the body 10 and of the release device 100 is illustrated in FIG. 5. Like component parts, features and functions are basically denoted in principle by like reference signs. To distinguish between the exemplary embodiment in FIG. 5 and that in FIGS. 1 to 4 however, the letter ‘a’ has been added to the reference signs of differently designed component parts in the exemplary embodiment of FIG. 5. The following description is basically restricted to the differences from the exemplary embodiment in FIGS. 1 to 4, wherein reference can be made to the description of the exemplary embodiment in FIGS. 1 to 4 with regard to like component parts, features and functions.

The body 10a or the release device 100a in FIG. 5 differs from the body 10 in FIGS. 1 to 4 in that the body 10a has an alternatively designed coupling element 26a. The coupling element 26a is formed by a toothing element 28 in the form of a toothed rack 72. The effective connection, which here is an interlocking connection, between the second grip segment 20a and a thread 24 is thus produced via the toothing element 28. The thread 24 itself may be a spindle 47 in this instance, formed on a main body 44a.

In addition, the body 10a in FIG. 5 differs in that the unlocking element 42a is formed in an alternative manner. The unlocking element 42a is formed separately from the second grip segment 20a and, as a mechanical operating element, comprises a ring 76 extending in the circumferential direction 22 about the second grip segment 20a. The toothing element 28 is arranged radially between the unlocking element 42a and the main body 44a in a through-opening 78 in the second grip segment 20a. The ring 76 is also arranged axially displacably in a radial indentation 80 in the second grip segment 20a. For a fast release of an implant (not shown here), the unlocking element 42a or the ring 76 is slid in the direction of a proximal end 12 of the body 10a. The movement of the unlocking element 42a occurs here against a spring bias of a spring element 40a, which biases the unlocking element 42a in its axial rest state. The spring element 40a is formed by a compression spring, which is fitted axially between a wall 68 of the through-opening 78 in the second grip segment 20a and an extension 82 of the unlocking element 42a extending in the radial direction 54 toward the inner axis 34. If the unlocking element 42a is then moved axially, a peg 84 integrally molded on the extension 82 runs along a slotted link 86. This slotted link 86, starting from an articulation point 88 of the peg 84 on the coupling element 26a, extends at an incline toward the inner axis 34. As a result of this movement of the peg 84, the coupling element 26a is moved radially away from an inner axis 34 of the insertion device 110 and therefore becomes disengaged from the thread 24, whereby the effective connection between the second grip segment 20a and the thread 24 can be released. In this case, a homogeneous movement of the coupling element 26a is achieved by the guidance of the coupling element by means of the walls 68a of the through-opening 78. The relative movement between the inner and outer insertion element 92, 94 is then ultimately caused by the fact that the second grip segment 20a, after the unlocking process, is drawn by the operator in the direction of the proximal end 12 of the body 10a.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

10 body

12 end

14 end

16 hand grip unit

18 grip segment

20 grip segment

22 circumferential direction

24 thread

26 coupling element

28 toothing element

30 connection element

32 fixing element

34 inner axis

36 cavity

38 transfer element

40 spring element

42 unlocking element

44 main body

46 axial direction

48 aperture

50 extension

52 groove

54 radial direction

56 depression

58 ball

60 ball cage

62 inner face

64 cavity

66 extension

68 wall

70 seat

72 toothed rack

74 spindle

76 ring

78 through-opening

80 indentation

82 extension

84 peg

86 slotted link

88 articulation point

90 shaft region

92 insertion element

94 insertion element

96 outer sleeve

100 release device

105 implant

107 end

109 end

110 insertion device

115 end

120 end

125 catheter tip

What is claimed is:

1. A release device for releasing a medical implant from an insertion device, with which the implant can be released by a relative movement between a first and a second insertion element, the release device comprising a body with a proximal end, which is remote from a distal end of the insertion device during use, and with a distal end, which faces the distal end of the insertion device during use, wherein a hand grip unit with a first grip segment fixed relative to the body and at least one movably arranged second grip segment is provided between the proximal and the distal end, wherein the movably arranged second grip segment is rotatable in the circumferential direction in order to effect an intended relative movement between the first and the second insertion element of the insertion device.

2. The release device as claimed in claim 1, wherein the second grip segment performs the relative movement between the first and the second insertion element of the insertion device via at least one effective connection to a thread.

3. The release device as claimed in claim 1, wherein the effective connection between the second grip segment and the thread is produced via at least one coupling element.

4. The release device as claimed in claim 1, wherein the effective connection between the second grip segment and the thread constitutes at least one force-locked connection.

5. The release device as claimed in claim 1, wherein the effective connection between the second grip segment and the thread is produced via at least one toothing element.

6. The release device as claimed in claim 1, wherein the relative movement between the first and the second insertion element of the insertion device is transferrable to one of the insertion elements by means of a connection element, wherein the connection element is effectively connected to the second grip segment.

7. The release device as claimed in claim 1, wherein the thread is fixed by means of at least one fixing element, which extends substantially radially in relation to an inner axis of the insertion device.

8. The release device as claimed in claim 6, wherein the connection element has at least one recess, in which the at least one fixing element engages in order to fix the connection element in the circumferential direction.

9. The release device as claimed in claim 6, comprising a transfer element, which is connected via a force-locked connection to the connection element, wherein the transfer element is arranged rotatably relative to the connection element.

10. The release device as claimed in claim 1, wherein the second grip segment is biased in its axial rest state by at least one spring element.

11. The release device as claimed in claim 2, wherein the effective connection between the second grip segment and the thread can be released by a movement of at least one unlocking element against a spring bias of at least one spring element.

12. The release device as claimed in claim 3, wherein, as a result of the movement of the at least one unlocking element, the at least one coupling element moves radially away from an inner axis of the insertion device and thus becomes disengaged from the thread.

13. The release device as claimed in claim 11, wherein the at least one unlocking element is formed in one piece with the second grip segment.

14. An insertion device for inserting a medical implant, which can be released by a relative movement between a first and a second insertion element, the insertion device comprising a release device for releasing the medical implant, in particular as claimed in claim 1, the release device comprising a body with a proximal end, which is remote from a distal end of the insertion device during use, and with a distal end, which faces the distal end of the insertion device during use, wherein a hand grip unit with a first grip segment fixed relative to the body and at least one movably arranged second grip segment is provided between the proximal and the distal end, wherein the movably arranged second grip segment is rotatable in the circumferential direction in order to effect an intended to relative movement between the first and the second insertion element of the insertion device.
